(19) United States
(12) Patent Application Publication
(10) Pub. No.: US 2013/0245060 A1
(43) Pub. Date: Sep. 19, 2013

(54) NOVEL COMPOSITION

(71) Applicants: Yanmin Hu, London (GB); Anthony Rm Coates, London (GB)

(72) Inventors: Yanmin Hu, London (GB); Anthony Rm Coates, London (GB)

(73) Assignee: HELPERBY THERAPEUTICS LIMITED, London (GB)

(21) Appl. No.: 13/824,986

(22) PCT Filed: Oct. 7, 2011
(86) PCT No.: PCT/GB2011/051931

§ 371 Date: Jun. 3, 2013

(30) Foreign Application Priority Data
Oct. 8, 2010 (GB) 1016999.3
May 10, 2011 (GB) 1107756.7

(51) Int. Cl. A61K 9/06; A61K 31/4745

(52) U.S. Cl. CPC A61K 9/06; A61K 31/4745
USPC 514/292

(57) ABSTRACT

This invention relates to topical pharmaceutical compositions comprising the active agent 4-methyl-1-(2-phenylethyl)-8-phenoxy-2,3-dihydro-1H-pyrrolo[3,2-c]-quinoline or a pharmaceutically acceptable derivative thereof and a hydrophobic excipient, to a process for preparing such compositions, and to the use of such compositions for the treatment of microbial infections.

This invention relates to topical pharmaceutical compositions comprising the active agent 4-methyl-1-(2-phenylethyl)-8-phenoxy-2,3-dihydro-1H-pyrrolo[3,2-c]-quinoline or a pharmaceutically acceptable derivative thereof and a hydrophobic excipient. Such compositions are useful for the treatment of microbial infections.

The synthesis and bactericidal activity of 4-methyl-1-(2-phenylethyl)-8-phenoxy-2,3-dihydro-1H-pyrrolo[3,2-c]-quinoline (I) is disclosed in International Patent Application, Publication Number WO2007054693

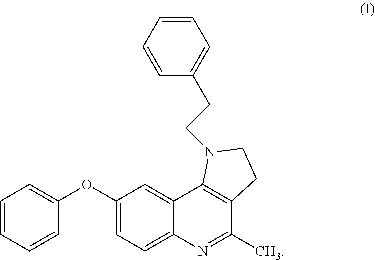 [SMILES: Cc1nc2ccc(Oc3ccccc3)cc2c2c1CCN2CCc1ccccc1]

International Patent Application, Publication Number WO2008056151 discloses topical compositions comprising a variety of pyrrolo[3,2-c]quinoline derivatives including 4-methyl-1-(2-phenylethyl)-8-phenoxy-2,3-dihydro-1H-pyrrolo[3,2-c]-quinoline. The examples given in this application are gel compositions that are characterised by a high (i.e. greater than 60% w/w) water or aqueous citrate/phosphate buffer content. Such compositions are comparatively stable, but on application are very readily absorbed to the systemic circulation, which limits their usefulness in the treatment of microbial infections that are resident on the surface of the skin or mucosal surfaces.

It is an object of the present invention to provide novel topical pharmaceutical compositions comprising 4-methyl-1-(2-phenylethyl)-8-phenoxy-2,3-dihydro-1H-pyrrolo[3,2-c]-quinoline or a pharmaceutically acceptable derivative thereof which are better suited to the treatment of microbial infections resident on the skin or mucosal surfaces than known compositions, and which maintain or improve the in vivo bactericidal potency of the active agent.

The present invention is based upon the unexpected finding that the surface residence time of topical compositions comprising 4-methyl-1-(2-phenylethyl)-8-phenoxy-2,3-dihydro-1H-pyrrolo[3,2-c]-quinoline or a pharmaceutically acceptable derivative thereof may be prolonged by the inclusion of one or more hydrophobic excipients therein, without compromising the antibacterial effect. Advantageously, certain compositions of the invention offer improved bactericidal activity compared to known compositions comprising 4-methyl-1-(2-phenylethyl)-8-phenoxy-2,3-dihydro-1H-pyrrolo[3,2-c]-quinoline.

The observation that the antibacterial activity of the compositions of the invention is retained or enhanced by the inclusion of one or more hydrophobic excipients therein is surprising since such excipients may be expected to reduce the bactericidal activity of a poorly soluble active ingredient such as 4-methyl-1-(2-phenylethyl)-8-phenoxy-2,3-dihydro-1H-pyrrolo[3,2-c]-quinoline, by retaining the drug in the formulation base.

Topical antibiotic compositions comprising paraffin-based hydrophobic excipients are known. For example, mupirocin calcium is commercially available as a nasal ointment under the trade name Bactroban® (GlaxoSmithKline). In addition to the active ingredient, this composition comprises white soft paraffin and Softisan 649®, a glycerine ester of natural fatty acids of isostearic acid and of adipic acid. Bactroban® is indicated for the elimination of nasal carriage staphylococci including methicillin-resistant Staphylococcus aureus (MRSA).

Walsh et al. (Pharmaceutical Research, 21(10), 1770-1775, 2004) have reported extended nasal residence times for topical compositions of lysostaphin and the monoclonal antibody BSYX-A110 comprising Softisan 649®, white petrolatum and paraffin.

In one embodiment, the present invention provides a topical pharmaceutical composition comprising 4-methyl-1-(2-phenylethyl)-8-phenoxy-2,3-dihydro-1H-pyrrolo[3,2-c]-quinoline or a pharmaceutically acceptable derivative thereof and a hydrophobic excipient.

In another embodiment, the present invention provides a topical pharmaceutical composition comprising 4-methyl-1-(2-phenylethyl)-8-phenoxy-2,3-dihydro-1H-pyrrolo[3,2-c]-quinoline or a pharmaceutically acceptable derivative thereof and a hydrophobic excipient for use in the treatment of a microbial infection.

In a further embodiment, the invention provides a method of treating a microbial infection which comprises administering to a mammal, including man, a topical pharmaceutical composition comprising 4-methyl-1-(2-phenylethyl)-8-phenoxy-2,3-dihydro-1H-pyrrolo[3,2-c]-quinoline or a pharmaceutically acceptable derivative thereof and a hydrophobic excipient.

In still a further embodiment, the invention provides the use of a topical pharmaceutical composition comprising 4-methyl-1-(2-phenylethyl)-8-phenoxy-2,3-dihydro-1H-pyrrolo[3,2-c]-quinoline or a pharmaceutically acceptable derivative thereof and a hydrophobic excipient for the treatment of a microbial infection.

As used herein, the term “hydrophobic excipient” means any pharmaceutically acceptable, substantially water-immiscible excipient that is capable of prolonging or extending the surface residence time of a topical composition comprising 4-methyl-1-(2-phenylethyl)-8-phenoxy-2,3-dihydro-1H-pyrrolo[3,2-c]-quinoline or a pharmaceutically acceptable derivative thereof. Suitably, the compositions of the invention exhibit a surface residence time (by visual inspection) of greater than 15 minutes, preferably greater than 30 minutes, following application to the skin or mucosal surface.

Suitable hydrophobic excipients include paraffin-based excipients or ointment and cream bases containing them. Such excipients are known in the art and/or are commercially available. Examples of suitable paraffin-based excipients include mixtures of solid and/or semi-solid saturated hydrocarbons having the general formula CₙH₂ₙ₊₂ obtainable from petroleum and/or shale oil, paraffin, white soft paraffin, liquid paraffin, light liquid paraffin and/or petrolatum, and mixtures thereof. Examples of suitable commercially available paraffin-containing ointment or cream bases include Unguentum M®, Paraffin Ointment BP, Simple Ointment BP and Emulsifying Ointment BP, and mixtures thereof. Examples of suitable commercially available petroleum-derived excipients include the MEKUR® and VARA® ranges sold by Sasol, such as MEKUR® 546, MEKUR® 500, MEKUR® 791, MEKUR® 773, VARA® 4800 and VARA® AB.

Other suitable hydrophobic excipients include “fixed” (vegetable based) oils such as almond oil, cottonseed oil, arachis oil, soy bean oil or their hydrogenated derivatives (such as hydrogenated cottonseed oil), cholesterol derivatives (such as Softisan®) and/or fatty acids (such as aluminium stearate), and mixtures thereof.

The hydrophobic excipient(s) is/are present in the compositions of the invention in an amount sufficient to prolong or extend the residence time of the composition when applied to the skin or mucosal surface. In one embodiment of the invention, the composition comprises from about 25 to about 99% (by weight of the total composition) of one or more hydrophobic excipients. Suitably, composition comprises from about 50 to about 98%, such as 50, 55, 60, 65, 70, 75, 80, 85, 90 or 95%, preferably from about 65 to about 90%, or from about 50 to about 75%, (by weight of the total composition) of one or more hydrophobic excipients.

The compositions of the present invention may be used to treat microbial infections. In particular they may be used to kill multiplying (i.e. log phase), non-multiplying (i.e. stationary phase) and/or clinically latent microorganisms associated with microbial infections. References herein to the treatment of a microbial infection therefore include killing multiplying non-multiplying and/or clinically latent microorganisms associated with such infections.

As used herein, “kill” means a loss of viability as assessed by a lack of metabolic activity.

As used herein, “clinically latent microorganism” means a microorganism that is metabolically active but has a growth rate that is below the threshold of infectious disease expression. The threshold of infectious disease expression refers to the growth rate threshold below which symptoms of infectious disease in a host are absent.

The metabolic activity of clinically latent microorganisms can be determined by several methods known to those skilled in the art; for example, by measuring mRNA levels in the microorganisms or by determining their rate of uridine uptake. In this respect, clinically latent microorganisms, when compared to microorganisms under logarithmic growth conditions (in vitro or in vivo), possess reduced but still significant levels of:
(I) mRNA (e.g. from 0.0001 to 50%, such as from 1 to 30, 5 to 25 or 10 to 20%, of the level of mRNA); and/or
(II) uridine (e.g. [³H]uridine) uptake (e.g. from 0.0005 to 50%, such as from 1 to 40, 15 to 35 or 20 to 30% of the level of [³H]uridine uptake).

Clinically latent microorganisms typically possess a number of identifiable characteristics. For example, they may be viable but non-culturable; i.e. they cannot typically be detected by standard culture techniques, but are detectable and quantifiable by techniques such as broth dilution counting, microscopy, or molecular techniques such as polymerase chain reaction. In addition, clinically latent microorganisms are phenotypically tolerant, and as such are sensitive (in log phase) to the biostatic effects of conventional antimicrobial agents (i.e. microorganisms for which the minimum inhibitory concentration (MIC) of a conventional antimicrobial is substantially unchanged); but possess drastically decreased susceptibility to drug-induced killing (e.g. microorganisms for which, with any given conventional antimicrobial agent, the ratio of minimum microbiocidal concentration (e.g. minimum bactericidal concentration, MBC) to MIC is 10 or more).

As used herein, the term “microorganisms” means fungi and bacteria. References herein to “microbial”, “antimicrobial” and “antimicrobially” shall be interpreted accordingly. For example, the term “microbial” means fungal or bacterial, and “microbial infection” means any fungal or bacterial infection.

As used herein, the term “bacteria” (and derivatives thereof, such as “microbial infection”) includes, but is not limited to, references to organisms (or infections due to organisms) of the following classes and specific types:

Gram-positive cocci, such as Staphylococci (e.g. Staph. aureus, Staph. epidermidis, Staph. saprophyticus, Staph. auricularis, Staph. capitis capitis, Staph. c. ureolyticus, Staph. caprae, Staph. cohnii cohnii, Staph. c. urealyticus, Staph. equorum, Staph. gallinarum, Staph. haemolyticus, Staph. hominis hominis, Staph. h. novobiosepticius, Staph. hyicus, Staph. intermedius, Staph. lugdunensis, Staph. pasteuri, Staph. saccharolyticus, Staph. schleiferi schleiferi, Staph. s. coagulans, Staph. sciuri, Staph. simulans, Staph. warneri and Staph. xylosus);

Streptococci (e.g. beta-haemolytic, pyogenic streptococci (such as Strept. agalactiae, Strept. canis, Strept dysgalactiae dysgalactiae, Strept dysgalactiae equisimilis, Strept equi equi, Strept equi zooepidemicus, Strept. iniae, Strept porcinus and Strept pyogenes), microaerophilic, pyogenic streptococci (Streptococcus “milleri”, such as Strept. anginosus, Strept constellatus constellatus, Strept constellatus pharyngidis and Strept intermedius), oral streptococci of the “mitis” (alpha-haemolytic—Streptococcus “viridans”, such as Strept. mitis, Strept. oralis, Strept. sanguinis, Strept. cristatus, Strept gordonii and Strept. parasanguinis), “salivarius” (non-haemolytic, such as Strept salivarius and Strept. vestibularis) and “mutans” (tooth-surface streptococci, such as Strept. criceti, Strept. mutans, Strept ratti and Strept. sobrinus) groups, Strept. acidominimus, Strept. bovis, Strept. faecalis, Strept. equinus, Strept. pneumoniae and Strept suis, or Streptococci alternatively classified as Group A, B, C, D, E, G, L, P, U or V Streptococcus);

Gram-negative cocci, such as Neisseria gonorrhoeae, Neisseria meningitidis, Neisseria cinerea, Neisseria elongata, Neisseria flavescens, Neisseria lactamica, Neisseria mucosa, Neisseria sicca, Neisseria subfiava and Neisseria weaveri;

Bacillaceae, such as Bacillus anthracis, Bacillus subtilis, Bacillus thuringiensis, Bacillus stearothermophilus and Bacillus cereus;

Enterobacteriaceae, such as Escherichia coli, Enterobacter (e.g. Enterobacter aerogenes, Enterobacter agglomerans and Enterobacter cloacae), Citrobacter (such as Citrob. freundii and Citrob. divernis), Hafnia (e.g. Hafnia alvei), Erwinia (e.g. Erwinia persicinus), Morganella morganii, Salmonella (Salmonella enterica and Salmonella typhi), Shigella (e.g. Shigella dysenteriae, Shigella flexneri, Shigella boydii and Shigella sonnei), Klebsiella (e.g. Klebs. pneumoniae, Klebs. oxytoca, Klebs. ornitholytica, Klebs. planticola, Klebs. ozaenae, Klebs. terrigena, Klebs. granulomatis (Calymmatobacterium granulomatis) and Klebs. rhinoscleromatis), Proteus (e.g. Pr. mirabilis, Pr. rettgeri and Pr. vulgaris), Providencia (e.g. Providencia alcalifaciens, Providencia rettgeri and Providencia stuartii), Serratia (e.g. Serratia marcescens and Serratia liquifaciens), and Yersinia (e.g. Yersinia enterocolitica, Yersinia pestis and Yersinia pseudotuberculosis);

Enterococci (e.g. Enterococcus avium, Enterococcus casseliflavus, Enterococcus cecorum, Enterococcus dispar, Enterococcus durans, Enterococcus faecalis, Enterococcus faecium, Enterococcus flavescens, Enterococcus gallinarum, Enterococcus hirae, Enterococcus malodoratus, Enterococcus mundtii, Enterococcus pseudoavium, Enterococcus raffinosus and Enterococcus solitarius);

Helicobacter (e.g. Helicobacter pylori, Helicobacter cinaedi and Helicobacter fennelliae);

Acinetobacter (e.g. A. baumanii, A. calcoaceticus, A. haemolyticus, A. johnsonii, A. junii, A. lwoffi and A. radioresistens);

Pseudomonas (e.g. Ps. aeruginosa, Ps. maltophilia (Stenotrophomonas maltophilia), Ps. alcaligenes, Ps. chlororaphis, Ps. fluorescens, Ps. luteola. Ps. mendocina, Ps. monteilii, Ps. oryzihabitans, Ps. pertocinogena, Ps. pseudalcaligenes, Ps. putida and Ps. stutzeri); Bacteriodes fragilis;

Peptococcus (e.g. Peptococcus niger);

Peptostreptococcus;

Clostridium (e.g. C. perfringens, C. difficile, C. botulinum, C. tetani, C. absonum, C. argentinense, C. baratii, C. bifermentans, C. beijerinckii, C. butyricum, C. cadaveris, C. camis, C. celatum, C. clostridioforme, C. cochlearium, C. cocleatum, C. fallax, C. ghonii, C. glycolicum, C. haemolyticum, C. hastiforme, C. histolyticum, C. indolis, C. innocuum, C. irregulare, C. leptum, C. limosum, C. malenominatum, C. novyi, C. oroticum, C. paraputrificum, C. piliforme, C. putrefasciens, C. ramosum, C. septicum, C. sordelii, C. sphenoides, C. sporogenes, C. subterminale, C. symbiosum and C. tedium);

Mycoplasma (e.g. M. pneumoniae, M. hominis, M. genitalium and M. urealyticum);

Mycobacteria (e.g. Mycobacterium tuberculosis, Mycobacterium avium, Mycobacterium fortuitum, Mycobacterium marinum, Mycobacterium kansasii, Mycobacterium chelonae, Mycobacterium abscesses, Mycobacterium leprae, Mycobacterium smegmitis, Mycobacterium africanum, Mycobacterium alvei, Mycobacterium asiaticum, Mycobacterium aurum, Mycobacterium bohemicum, Mycobacterium bovis, Mycobacterium branderi, Mycobacterium brumae, Mycobacterium celatum, Mycobacterium chubense, Mycobacterium confluentis, Mycobacterium conspicuum, Mycobacterium cookii, Mycobacterium flavescens, Mycobacterium gadium, Mycobacterium gastri, Mycobacterium genavense, Mycobacterium gordonae, Mycobacterium goodii, Mycobacterium haemophilum, Mycobacterium hassicum, Mycobacterium intracellulare, Mycobacterium interjectum, Mycobacterium heidelberense, Mycobacterium lentiflavum, Mycobacterium malmoense, Mycobacterium mucogenicum, Mycobacterium microti, Mycobacterium mucogenicum, Mycobacterium neoaurum, Mycobacterium nonchromogenicum, Mycobacterium peregrinum, Mycobacterium phlei, Mycobacterium scrofulaceum, Mycobacterium shimoidei, Mycobacterium simiae, Mycobacterium szulgai, Mycobacterium terrae, Mycobacterium thermoresistabile, Mycobacterium triplex, Mycobacterium triviale, Mycobacterium tusciae, Mycobacterium ulcerans, Mycobacterium vaccae, Mycobacterium wolinskyi and Mycobacterium xenopi);

Haemophilus (e.g. Haemophilus influenzae, Haemophilus ducreyi, Haemophilus aegyptius, Haemophilus parainfluenzae, Haemophilus haemolyticus and Haemophilus parahaemolyticus);

Actinobacillus (e.g. Actinobacillus actinomycetemcomitans, Actinobacillus equuli, Actinobacillus hominis, Actinobacillus lignieresii, Actinobacillus suis and Actinobacillus ureae);

Actinomyces (e.g. Actinomyces israelii);

Brucella (e.g. Brucella abortus, Brucella canis, Brucella melintensis and Brucella suis);

Campylobacter (e.g. Campylobacter jejuni, Campylobacter coli, Campylobacter lari and Campylobacter fetus);

Listeria monocytogenes;

Vibrio (e.g. Vibrio cholerae and Vibrio parahaemolyticus, Vibrio alginolyticus, Vibrio carchariae, Vibrio fluvialis, Vibrio furnissii, Vibrio hollisae, Vibrio metschnikovii, Vibrio mimicus and Vibrio vulnificus);

Erysipelothrix rhusopathiae;

Corynebacteriaceae (e.g. Corynebacterium diphtheriae, Corynebacterium jeikeum and Corynebacterium urealyticum);

Spirochaetaceae, such as Borrelia (e.g. Borrelia recurrentis, Borrelia burgdorferi, Borrelia afzelii, Borrelia andersonii, Borrelia bissettii, Borrelia garinii, Borrelia japonica, Borrelia lusitaniae, Borrelia tanukii, Borrelia turdi, Borrelia valaisiana, Borrelia caucasica, Borrelia crocidurae, Borrelia duttoni, Borrelia graingeri, Borrelia hermsii, Borrelia hispanica, Borrelia latyschewii, Borrelia mazzottii, Borrelia parkeri, Borrelia persica, Borrelia turicatae and Borrelia venezuelensis) and Treponema (Treponema pallidum ssp. pallidum, Treponema pallidum ssp. endemicum, Treponema pallidum ssp. pertenue and Treponema carateum);

Pasteurella (e.g. Pasteurella aerogenes, Pasteurella bettyae, Pasteurella canis, Pasteurella dagmatis, Pasteurella gallinarum, Pasteurella haemolytica, Pasteurella multocida multocida, Pasteurella multocida gallicida, Pasteurella multocida septica, Pasteurella pneumotropica and Pasteurella stomatis); Bordetella (e.g. Bordetella bronchiseptica, Bordetella hinzii, Bordetella holmseii, Bordetella parapertussis, Bordetella pertussis and Bordetella trematum);

Nocardiaceae, such as Nocardia (e.g. Nocardia asteroides and Nocardia brasiliensis);

Rickettsia (e.g. Ricksettsii or Coxiella bumetii);

Legionella (e.g. Legionalla anisa, Legionalla birminghamensis, Legionalla bozemanii, Legionalla cincinnatiensis, Legionalla dumoffii, Legionalla feeleii, Legionalla gormanii, Legionalla hackeliae, Legionalla israelensis, Legionalla jordanis, Legionalla lansingensis, Legionalla longbeachae, Legionalla maceachernii, Legionalla micdadei, Legionalla oakridgensis, Legionalla pneumophila, Legionalla sainthelensi, Legionalla tucsonensis and Legionalla wadsworthii);

Moraxella catarrhalis;

Cyclospora cayetanensis;

Entamoeba histolytica;

Giardia lamblia;

Trichomonas vaginalis;

Toxoplasma gondii;

Stenotrophomonas maltophilia;

Burkholderia cepacia; Burkholderia mallei and Burkholderia pseudomallei;

Francisella tularensis;

Gardnerella (e.g. Gardneralla vaginalis and Gardneralla mobiluncus);

Streptobacillus moniliformis;

Flavobacteriaceae, such as Capnocytophaga (e.g. Capnocytophaga canimorsus, Capnocytophaga cynodegmi, Capnocytophaga gingivalis, Capnocytophaga granulosa, Capnocytophaga haemolytica, Capnocytophaga ochracea and Capnocytophaga sputigena);

Bartonella (Bartonella bacilliformis, Bartonella clarridgeiae, Bartonella elizabethae, Bartonella henselae, Bartonella quintana and Bartonella vinsonii arupensis);

Leptospira (e.g. Leptospira biflexa, Leptospira borgpetersenii, Leptospira inadai, Leptospira interrogans, Leptospira kirschneri, Leptospira noguchii, Leptospira santarosai and Leptospira weilii);

Spirillium (e.g. Spirillum minus);

Baceteroides (e.g. Bacteroides caccae, Bacteroides capillosus, Bacteroides coagulans, Bacteroides distasonis, Bacteroides eggerthii, Bacteroides forsythus, Bacteroides fragilis, Bacteroides merdae, Bacteroides ovatus, Bacteroides putredinis, Bacteroides pyogenes, Bacteroides splanchinicus, Bacteroides stercoris, Bacteroides tectus, Bacteroides thetaiotaomicron, Bacteroides uniformis, Bacteroides ureolyticus and Bacteroides vulgatus);

Prevotella (e.g. Prevotella bivia, Prevotella buccae, Prevotella corporis, Prevotella dentalis (Mitsuokella dentalis), Prevotella denticola, Prevotella disiens, Prevotella enoeca, Prevotella heparinolytica, Prevotella intermedia, Prevotella loeschii, Prevotella melaminogenica, Prevotella nigrescens, Prevotella oralis, Prevotella oris, Prevotella oulora, Prevotella tannerae, Prevotella venoralis and Prevotella zoogleoformans);

Porphyromonas (e.g. Porphyromonas asaccharolytica, Porphyromonas cangingivalis, Porphyromonas canoris, Porphyromonas cansulci, Porphyromonas catoniae, Porphyromonas circumdentaria, Porphyromonas crevioricanis, Porphyromonas endodontalis, Porphyromonas gingivalis, Porphyromonas gingivicanis, Porphyromonas levii and Porphyromonas macacae);

Fusobacterium (e.g. F. gonadiaformans, F. mortiferum, F. naviforme, F. necrogenes, F. necrophorum necrophorum, F. necrophorum fundiliforme, F. nucleatum nucleatum, F. nucleatum fusiforme, F. nucleatum polymorphum, F. nucleatum vincentii, F. periodonticum, F. russii, F. ulcerans and F. varium);

Chlamydia (e.g. Chlamydia trachomatis);

Cryptosporidium (e.g. C. parvum, C. hominis, C. canis, C. fells, C. meleagridis and C. muris);

Chlamydophila (e.g. Chlamydophila abortus (Chlamydia psittaci), Chlamydophila pneumoniae (Chlamydia pneumoniae) and Chlamydophila psittaci (Chlamydia psittaci));

Leuconostoc (e.g. Leuconostoc citreum, Leuconostoc cremoris, Leuconostoc dextranicum, Leuconostoc lactis, Leuconostoc mesenteroides and Leuconostoc pseudomesenteroides);

Gemella (e.g. Gemella bergeri, Gemella haemolysans, Gemella morbillorum and Gemella sanguinis); and

Ureaplasma (e.g. Ureaplasma parvum and Ureaplasma urealyticum).

As used herein, the term “fungi” (and derivatives thereof, such as “fungal infection”) includes, but is not limited to, references to organisms (or infections due to organisms) of the following classes and specific types:

Absidia (e.g. Absidia corymbifera);

Ajellomyces (e.g. Ajellomyces capsulatus and Ajellomyces dermatitidis);

Arthroderma (e.g. Arthroderma benhamiae, Arthroderma fulvum, Arthroderma gypseum, Arthroderma incurvatum, Arthroderma otae and Arthroderma vanbreuseghemii);

Aspergillus (e.g. Aspergillus flavus, Aspergillus fumigatus and Aspergillus niger);

Blastomyces (e.g. Blastomyces dermatitidis);

Candida (e.g. Candida albicans, Candida glabrata, Candida guilliermondii, Candida krusei, Candida parapsilosis, Candida tropicalis and Candida pelliculosa);

Cladophialophora (e.g. Cladophialophora carrionii);

Coccidioides (e.g. Coccidioides immitis and Coccidioides posadasii);

Cryptococcus (e.g. Cryptococcus neoformans);

Cunninghamella (e.g. Cunninghamella sp.) Epidermophyton (e.g. Epidermophyton floccosum);

Exophiala (e.g. Exophiala dermatitidis);

Filobasidiella (e.g. Filobasidiella neoformans);

Fonsecaea (e.g. Fonsecaea pedrosoi);

Fusarium (e.g. Fusarium solani);

Geotrichum (e.g. Geotrichum candidum);

Histoplasma (e.g. Histoplasma capsulatum);

Hortaea (e.g. Hortaea wemeckil);

Issatschenkia (e.g. Issatschenkia orientalis);

Madurella (e.g. Madurella grisae);

Malassezia (e.g. Malassezia furfur, Malassezia globosa, Malassezia obtusa, Malassezia pachydermatis, Malassezia restricta, Malassezia slooffiae and Malassezia sympodialis);

Microsporum (e.g. Microsporum canis, Microsporum fulvum and Microsporum gypseum);

Microsporidia;

Mucor (e.g. Mucor circinelloides);

Nectria (e.g. Nectria haematococca);

Paecilomyces (e.g. Paecilomyces variotii);

Paracoccidioides (e.g. Paracoccidioides brasiliensis);

Penicillium (e.g. Penicillium marneffei);

Pichia (e.g. Pichia anomala and Pichia guilliermondii);

Pneumocystis (e.g. Pneumocystis jiroveci (Pneumocystis carinii));

Pseudallescheria (e.g. Pseudallescheria boydii);

Rhizopus (e.g. Rhizopus oryzae);

Rhodotorula (e.g. Rhodotorula rubra);

Scedosporium (e.g. Scedosporium apiospermum);

Schizophyllum (e.g. Schizophyllum commune);

Sporothrix (e.g. Sporothrix schenckii);

Trichophyton (e.g. Trichophyton mentagrophytes, Trichophyton rubrum, Trichophyton verrucosum and Trichophyton violaceum); and

Trichosporon (e.g. Trichosporon asahii, Trichosporon cutaneum, Trichosporon inkin and Trichosporon mucoides).

Particular bacteria that may treated using a composition of the invention include:

Staphylococci, such as Staph. aureus (either Methicillin-sensitive (i.e. MSSA) or Methicillin-resistant (i.e. MRSA)) and Staph. epidermidis;

Streptococci, such as Strept. agalactiae and Strept. pyogenes;

Bacillaceae, such as Bacillus anthracis;

Enterobacteriaceae, such as Escherichia coli, Klebsiella (e.g. Klebs. pneumoniae and Klebs. oxytoca) and Proteus (e.g. Pr. mirabilis, Pr. rettgeri and Pr. vulgaris);

Haemophilis influenzae;

Enterococci, such as Enterococcus faecalis and Enterococcus faecium; and

Mycobacteria, such as Mycobacterium tuberculosis.

Preferably, the bacterium is Staph. Aureus; either MSSA or MRSA.

Particular fungi that may be treated using a composition of the invention include Aspergillus fumigatus, Candida albicans, Cryptococcus neoformans, Histoplasma capsulatum and Pneumocystis jiroveci.

The compositions of the present invention may be used to treat infections associated with any one or more of the above-mentioned bacterial or fungal organisms, and in particular they may be used for killing multiplying, non-multiplying and/or clinically latent microorganisms associated with such an infection.

Particular conditions which may be treated using the compositions of the present invention include tuberculosis (e.g. pulmonary tuberculosis, non-pulmonary tuberculosis (such as tuberculosis lymph glands, genito-urinary tuberculosis, tuberculosis of bone and joints, tuberculosis meningitis) and miliary tuberculosis), anthrax, abscesses, acne vulgaris, actinomycosis, asthma, bacilliary dysentry, bacterial conjunctivitis, bacterial keratitis, bacterial vaginosis, botulism, Buruli ulcer, bone and joint infections, bronchitis (acute or chronic), brucellosis, burn wounds, cat scratch fever, cellulitis, chancroid, cholangitis, cholecystitis, cutaneous diphtheria, cystic fibrosis, cystitis, diffuse panbronchiolitis, diphtheria, dental caries, diseases of the upper respiratory tract, eczema, empymea, endocarditis, endometritis, enteric fever, enteritis, epididymitis, epiglottitis, erysipelis, erysipclas, erysipeloid, erythrasma, eye infections, furuncles, gardnerella vaginitis, gastrointestinal infections (gastroenteritis), genital infections, gingivitis, gonorrhoea, granuloma inguinale, Haverhill fever, infected burns, infections following dental operations, infections in the oral region, infections associated with prostheses, intraabdominal abscesses, Legionnaire's disease, leprosy, leptospirosis, listeriosis, liver abscesses, Lyme disease, lymphogranuloma venerium, mastitis, mastoiditis, meningitis and infections of the nervous system, mycetoma, nocardiosis (e.g. Madura foot), non-specific urethritis, opthalmia (e.g. opthalmia neonatorum), osteomyelitis, otitis (e.g. otitis externa and otitis media), orchitis, pancreatitis, paronychia, pelveoperitonitis, peritonitis, peritonitis with appendicitis, pharyngitis, phlegmons, pinta, plague, pleural effusion, pneumonia, postoperative wound infections, postoperative gas gangrene, prostatitis, pseudo-membranous colitis, psittacosis, pulmonary emphysema, pyelonephritis, pyoderma (e.g. impetigo), Q fever, rat-bite fever, reticulosis, ricin poisoning, Ritter's disease, salmonellosis, salpingitis, septic arthritis, septic infections, septicameia, sinusitis, skin infections (e.g. skin granulomas, impetigo, folliculitis and furunculosis), syphilis, systemic infections, tonsillitis, toxic shock syndrome, trachoma, tularaemia, typhoid, typhus (e.g. epidemic typhus, murine typhus, scrub typhus and spotted fever), urethritis, wound infections, yaws, aspergillosis, candidiasis (e.g. oropharyngeal candidiasis, vaginal candidiasis or balanitis), cryptococcosis, favus, histoplasmosis, intertrigo, mucormycosis, tinea (e.g. tinea corporis, tinea capitis, tinea cruris, tinea pedis and tinea unguium), onychomycosis, pityriasis versicolor, ringworm and sporotrichosis; or infections with MSSA, MRSA, Staph. epidermidis, Strept. agalactiae, Strept. pyogenes, Escherichia coli, Klebs. pneumoniae, Klebs. oxytoca, Pr. mirabilis, Pr. rettgeri, Pr. vulgaris, Haemophilis influenzae, Enterococcus faecalis and Enterococcus faecium.

The topical pharmaceutical compositions of the present invention may be used to treat a variety of skin or membrane disorders, such as infections of the skin or membranes (e.g. infections of nasal membranes, axilla, groin, perineum, rectum, dermatitic skin, skin ulcers, and sites of insertion of medical equipment such as i.v. needles, catheters and tracheostomy or feeding tubes) with any of the bacteria, fungi described above, (e.g. any of the Staphylococci, Streptococci, Mycobacteria or Pseudomonas organisms mentioned hereinbefore, such as S. aureus (e.g. Methicillin resistant S. aureus (MRSA))).

Particular bacterial conditions that may be treated by topical pharmaceutical compositions of the present invention also include the skin- and membrane-related conditions disclosed hereinbefore, as well as: acne vulgaris; rosacea (including erythematotelangiectatic rosacea, papulopustular rosacea, phymatous rosacea and ocular rosacea); erysipelas; erythrasma; eethyma; eethyma gangrenosum; impetigo; paronychia; cellulitis; folliculitis (including hot tub folliculitis); furunculosis; carbunculosis; staphylococcal scalded skin syndrome; surgical scarlet fever; streptococcal peri-anal disease; streptococcal toxic shock syndr ome; pitted keratolysis; trichomycosis axillaris; pyoderma; external canal ear infections; green nail syndrome; spirochetes; necrotizing fasciitis; Mycobacterial skin infections (such as lupus vulgaris, scrofuloderma, warty tuberculosis, tuberculides, erythema nodosum, erythema induratum, cutaneous manifestations of tuberculoid leprosy or lepromatous leprosy, erythema nodosum leprosum, cutaneous M. kansasii, M. malmoense, M. szulgai, M. simiae, M. gordonae, M. haemophilum, M. avium, M. intracellulare, M. chelonae (including M. abscessus) or M. fortuitum infections, swimming pool (or fish tank) granuloma, lymphadenitis and Buruli ulcer (Bairnsdale ulcer, Searles' ulcer, Kakerifu ulcer or Toro ulcer)); as well as infected eczma, burns, abrasions and skin wounds.

In a preferred embodiment of the invention there is provided the use of a topical pharmaceutical composition comprising 4-methyl-1-(2-phenylethyl)-8-phenoxy-2,3-dihydro-1H-pyrrolo[3,2-c]-quinoline or a pharmaceutically acceptable derivative thereof and a hydrophobic excipient for nasal decolonisation of MSSA or MRSA, preferably MRSA.

Particular fungal conditions that may be treated by topical pharmaceutical compositions of the present invention also include the skin- and membrane-related conditions disclosed hereinbefore, as well as: candidiasis; sporotrichosis; ringworm (e.g. tinea pedis, tinea cruris, tinea capitis, tinea unguium or tinea corporis); tinea versicolor; and infections with Trichophyton, Microsporum, Epidermophyton or Pityrosporum ovale fungi.

It will be appreciated that references herein to “treatment” extend to prophylaxis as well as the treatment of established diseases or symptoms.

The compositions of the invention may be administered in combination with one or more additional compounds that possess bactericidal activity.

As used herein, the term “in combination with” covers separate, simultaneous and sequential administration of 4-methyl-1-(2-phenylethyl)-8-phenoxy-2,3-dihydro-1H-pyrrolo[3,2-c]-quinoline or a pharmaceutically acceptable derivative thereof and one or more additional antimicrobial agents. When the agents are administered sequentially, either 4-methyl-1-(2-phenylethyl)-8-phenoxy-2,3-dihydro-1H-pyrrolo[3,2-c]-quinoline or a pharmaceutically acceptable derivative thereof or an additional antimicrobial agent may be administered first. When administration is simultaneous, the agents may be administered either in the same or a different pharmaceutical composition. Adjunctive therapy, i.e. where one agent is used as a primary treatment and the other agent is used to assist that primary treatment, is also an embodiment of the present invention.

Suitable additional antimicrobial agents for use in the present invention include one or more compounds selected from the following:
(1) 6-Lactams, including:
   (i) penicillins, such as
      (I) benzylpenicillin, procaine benzylpenicillin, phenoxy-methylpenicillin, methicillin, propicillin, epicillin, cyclacillin, hetacillin, 6-aminopenicillanic acid, penicillic acid, penicillanic acid sulphone (sulbactam), penicillin G, penicillin V, phenethicillin, phenoxymethylpenicillinic acid, azlocillin, carbenicillin, cloxacillin, D-(−)-penicillamine, dicloxacillin, nafcillin and oxacillin,
      (II) penicillinase-resistant penicillins (e.g. flucloxacillin),
      (III) broad-spectrum penicillins (e.g. ampicillin, amoxicillin, metampicillin and bacampicillin),
      (IV) antipseudomonal penicillins (e.g. carboxypenicillins such as ticarcillin or ureidopenicillins such as piperacillin),
      (V) mecillinams (e.g. pivmecillinam), or
      (VI) combinations of any two or more of the agents mentioned at (I) to (V) above, or combinations of any of the agents mentioned at (I) to (V) above with a β-lactamase inhibitor such as tazobactam or, particularly, clavulanic acid (which acid is optionally in metal salt form, e.g. in salt form with an alkali metal such as sodium or, particularly, potassium);
   (ii) cephalosporins, such as cefaclor, cefadroxil, cefalexin (cephalexin), cefcapene, cefcapene pivoxil, cefdinir, cefditoren, cefditoren pivoxil, cefixime, cefotaxime, cefpirome, cefpodoxime, cefpodoxime proxetil, cefprozil, cefradine, ceftazidime, cefteram, cefteram pivoxil, ceftriaxone, cefuroxime, cefuroxime axetil, cephaloridine, cephacetrile, cephamandole, cephaloglycine, ceftobiprole, PPI-0903 (TAK-599), 7-aminocephalosporanic acid, 7-aminodes-acetoxycephalosporanic acid, cefamandole, cefazolin, cefinetazole, cefoperazone, cefsulodin, cephalosporin C zinc salt, cephalothin, cephapirin; and
   (iii) other β-lactams, such as monobactams (e.g. aztreonam), carbapenems (e.g. imipenem (optionally in combination with a renal enzyme inhibitor such as cilastatin), meropenem, ertapenem, doripenem (S-4661) and R04908463 (CS-023)), penems (e.g. faropenem) and 1-oxa-β-lactams (e.g. moxalactam).
(2) Tetracyclines, such as tetracycline, demeclocycline, doxycycline, lymecycline, minocycline, oxytetracycline, chlortetracycline, meclocycline and methacycline, as well as glycylcyclines (e.g. tigecycline).
(3) Aminoglycosides, such as amikacin, gentamicin, netilmicin, neomycin, streptomycin, tobramycin, amastatin, butirosin, butirosin A, daunorubicin, dibekacin, dihydrostreptomycin, G 418, hygromycin B, kanamycin B, kanamycin, kirromycin, paromomycin, ribostamycin, sisomicin, spectinomycin, streptozocin and thiostrepton.
(4) (i) Macrolides, such as azithromycin, clarithromycin, erythromycin, roxithromycin, spiramycin, amphotericins B (e.g. amphotericin B), bafilomycins (e.g. bafilomycin A1), brefeldins (e.g. brefeldin A), concanamycins (e.g. concanamycin A), filipin complex, josamycin, mepartricin, midecamycin, nonactin, nystatin, oleandomycin, oligomycins (e.g. oligomycin A, oligomycin B and oligomycin C), pimaricin, rifampicin, rifamycin, rosamicin, tylosin, virginiamycin and fosfomycin.
   (ii) Ketolides such as telithromycin and cethromycin (ABT-773).
   (iii) Lincosamines, such as lincomycin.
(5) Clindamycin and clindamycin 2-phosphate.
(6) Phenicols, such as chloramphenicol and thiamphenicol.
(7) Steroids, such as fusidic acid (optionally in metal salt form, e.g. in salt form with an alkali metal such as sodium).
(8) Glycopeptides such as vancomycin, teicoplanin, bleomycin, phleomycin, ristomycin, telavancin, dalbavancin and oritavancin.
(9) Oxazolidinones, such as linezolid and AZD2563.
(10) Streptogramins, such as quinupristin and dalfopristin, or a combination thereof.
(11) (i) Peptides, such as polymyxins (e.g. colistin and polymyxin B), lysostaphin, duramycin, actinomycins (e.g. actinomycin C and actinomycin D), actinonin, 7-aminoactinomycin D, antimycin A, antipain, bacitracin, cyclosporin A, echinomycin, gramicidins (e.g. gramicidin A and gramicidin C), myxothiazol, nisin, paracelsin, valinomycin and viomycin.
   (ii) Lipopeptides, such as daptomycin.
   (iii) Lipoglycopeptides, such as ramoplanin.
(12) Sulfonamides, such as sulfamethoxazole, sulfadiazine, sulfaquinoxaline, sulfathiazole (which latter two agents are optionally in metal salt form, e.g. in salt form with an alkali metal such as sodium), succinylsulfathiazole, sulfadimethoxine, sulfaguanidine, sulfamethazine, sulfamonomethoxine, sulfanilamide and sulfasalazine.
(13) Trimethoprim, optionally in combination with a sulfonamide, such as sulfamethoxazole (e.g. the combination co-trimoxazole).
(14) Antituberculous drugs, such as isoniazid, rifampicin, rifabutin, pyrazinamide, ethambutol, streptomycin, amikacin, capreomycin, kanamycin, quinolones (e.g. those at (q) below), para-aminosalicylic acid, cycloserine and ethionamide.
(15) Antileprotic drugs, such as dapsone, rifampicin and clofazimine.
(16) (i) Nitroimidazoles, such as metronidazole and timidazole.
   (ii) Nitrofurans, such as nitrofurantoin.
(17) Quinolones, such as nalidixic acid, norfloxacin, ciprofloxacin, ofloxacin, levofloxacin, moxifloxacin, gatifloxacin, gemifloxacin, garenoxacin, DX-619, WCK 771 (the arginine salt of S-(−)-nadifloxacin), 8-quinolinol, cinoxacin, enrofloxacin, flumequine, lomefloxacin, oxolinic acid and pipemidic acid.
(18) Amino acid derivatives, such as azaserine, bestatin, D-cycloserine, 1,10-phenanthroline, 6-diazo-5-oxo-L-norleucine and L-alanyl-L-1-aminoethyl-phosphonic acid.
(19) Aureolic acids, such as chromomycin A3, mithramycin A and mitomycin C.
(20) Benzochinoides, such as herbimycin A.
(21) Coumarin-glycosides, such as novobiocin.
(22) Diphenyl ether derivatives, such as irgasan.
(23) Epipolythiodixopiperazines, such as gliotoxin from Gliocladium fimbriatum.
(24) Fatty acid derivatives, such as cerulenin.
(25) Glucosamines, such as 1-deoxymannojirimycin, 1-deoxynojirimycin and N-methyl-1-deoxynojirimycin.
(26) Indole derivatives, such as staurosporine.
(27) Diaminopyrimidines, such as iclaprim (AR-100).
(28) Macrolactams, such as ascomycin.
(29) Taxoids, such as paclitaxel.
(30) Statins, such as mevastatin.
(31) Polyphenolic acids, such as (+)-usnic acid.
(32) Polyethers, such as lasalocid A, lonomycin A, monensin, nigericin and salinomycin.
(33) Picolinic acid derivatives, such as fusaric acid.
(34) Peptidyl nucleosides, such as blasticidine S, nikkomycin, nourseothricin and puromycin.
(35) Nucleosides, such as adenine 9-β-D-arabinofuranoside, 5-azacytidine, cordycepin, formycin A, tubercidin and tunicamycin.
(36) Pleuromutilins, such as GSK-565154, GSK-275833 and tiamulin.
(37) Peptide deformylase inhibitors, such as LBM415 (NVP PDF-713) and BB 83698.
(38) Antibacterial agents for the skin, such as fucidin, benzamycin, clindamycin, erythromycin, tetracycline, silver sulfadiazine, chlortetracycline, metronidazole, mupirocin, framycitin, gramicidin, neomycin sulfate, polymyxins (e.g. polymixin B) and gentamycin.
(39) Miscellaneous agents, such as methenamine (hexamine), doxorubicin, piericidin A, stigmatellin, actidione, anisomycin, apramycin, coumermycin A1, L(+)-lactic acid, cytochalasins (e.g. cytochalasin B and cytochalasin D), emetine and ionomycin.
(40) Antiseptic agents, such as chlorhexidine, phenol derivatives (e.g. thymol and triclosan), quarternary ammonium compounds (e.g. benzalkonium chloride, cetylpyridinium chloride, benzethonium chloride, cetrimonium bromide, cetrimonium chloride and cetrimonium stearate), octenidine dihydrochloride, and terpenes (e.g. terpinen-4-ol).

4-methyl-1-(2-phenylethyl)-8-phenoxy-2,3-di hydro-1H-pyrrolo[3,2-c]-q uinoline and pharmaceutically acceptable derivatives thereof may be prepared according to the methods disclosed in International Patent Application, Publication Numbers WO2007054693 and WO2008056151. The contents of these documents are incorporated herein by reference as if each individual publication was specifically and fully set forth herein.

As used herein the term “pharmaceutically acceptable derivative” means:
(a) pharmaceutically acceptable salts with either acids or bases (e.g. acid addition salts); and/or
(b) solvates (including hydrates).

Acid addition salts that may be mentioned include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, hemi-tartrate, tartrate, phenylacetate, mandelate, phenylpropionate, phenyl butyrate, benzoate, chlorobenzoate, methylbenzoate, hydroxybenzoate, methoxybenzoate, dinitrobenzoate, o-acetoxybenzoate, salicylate, nicotinate, isonicotinate, cinnamate, oxalate, malonate, hemi-succinate, succinate, suberate, sebacate, fumarate, malate, maleate, hydroxymaleate, hippurate, phthalate or terephthalate salts), halide salts (e.g. chloride, bromide or iodide salts), sulfonate salts (e.g. benzenesulfonate, methyl-, bromo- or chloro-benzenesulfonate, xylenesulfonate, p-toluene sulfonate (tosylate), methane sulfonate (mesylate), ethanesulfonate, propanesulfonate, hydroxyethanesulfonate, 1- or 2-naphthalene-sulfonate or 1,5-naphthalenedisulfonate salts) or sulfate, pyrosulfate, bisulfate, sulfite, bisulfite, phosphate, monohydrogenphosphate, dihydrogenphosphate, metaphosphate, pyrophosphate or nitrate salts, and the like.

Preferably, the acid addition salt of 4-methyl-1-(2-phenylethyl)-8-phenoxy-2,3-dihydro-1H-pyrrolo[3,2-c]-quinoline selected from the group consisting of the hydrobromic acid, hydrochloric acid, methane sulphonic acid, p-toluene sulphonic acid, succinic acid (preferably hemi-succinate), sulphuric acid and tartaric acid (preferably hemi-tartrate) addition salts thereof. Most preferably the acid addition salt is 4-methyl-1-(2-phenylethyl)-8-phenoxy-2,3-dihydro-1H-pyrrolo[3,2-c]-quinoline hydrochloride or methane sulfonate.

Acid addition salts of 4-methyl-1-(2-phenylethyl)-8-phenoxy-2,3-dihydro-1H-pyrrolo[3,2-c]-quinoline may be prepared by conventional methods known in the art, for example as described in Berge, S. M. et al., J. Pharm. Sci., 1977, 66(1), 1-19; Stahl, P. H. and Wermuth, C. G., Handbook of Pharmaceutical Salts: Properties, Selection and Use, 2011, 2^{nd} Edition, Wiley-VCH, and references cited therein.

The compositions of the present invention comprise one or more hydrophobic excipients. In a preferred embodiment of the invention, the hydrophobic excipient is selected from the group consisting of Unguentum M®, Emulsifying Ointment BP, liquid paraffin and mixtures thereof. In a further preferred embodiment of the invention, the hydrophobic excipient is Unguentum M® or a mixture of Emulsifying Ointment BP and liquid paraffin. In still a further preferred embodiment of the invention, the hydrophobic excipient is a petroleum jelly (for example, MEKUR® 773), a cholesterol derivative (for example, Softisan®) or a mixture thereof.

Suitably, Unguentum M® is present in the composition in an amount of from about 50 to about 75% (w/w), preferably in an amount from about 60 to about 70% (w/w), for example about 55, 60, 65, 70 or 75% (w/w) (by weight of the total composition).

Suitably, Emulsifying Ointment BP is present in the composition in an amount of from about 50 to about 75%, for example about 55, 60, 65, 70 or 75% (w/w), and more preferably about 63% (w/w) (by weight of the total composition).

Suitably, liquid paraffin is present in the composition in an amount of from about 20 to about 40%, for example about 20, 25, 30, 35 or 40% (w/w), more preferably about 30% (w/w) (by weight of the total composition).

Suitably, petroleum jelly (for example MEKUR® 773) is present in the composition in an amount of from about 20 to about 75%, for example about 20, 25, 30, 35, 40, 45, 50, 55, 60, 65, 70 or 75% (w/w), more preferably about 35 to about 60% (w/w) (by weight of the total composition).

Suitably, a cholesterol derivative (for example, Softisan®, such as Softisan® 649) is present in the composition in an amount of from about 20 to about 40%, for example about 20, 25, 30, 35 or 40%, more preferably about 30% (w/w) (by weight of the total composition).

In addition to one or more hydrophobic excipients, the compositions of the present invention may also comprise one or more additional excipients selected from the group consisting of emulsifiers, emulsion stabilisers, solubilising agents, solvents, thickening agents, gelling agents, and/or preservatives.

Examples of suitable emulsifiers include polyethylene glycol ethers (such as Cetomacragol 1000), fatty acid polyhydric alcohol esters (such as sorbitan mono-oleate) and polyethylene glycol ethers thereof (such as Polysorbate 80), and ethylene glycol palmitostearate, and mixtures thereof.

Examples of suitable emulsion stabilisers include cetostearyl alcohol, cetyl esters, cholesterol, dibutyl sebacate, dimethicone, glycerine, glycerin monostearate, glyceryl monooleate, glyceryl monostearate, isopropyl myristate, isopropyl palmitate, lanolin and lecithin, and mixtures thereof.

In one embodiment of the invention, the composition comprises an emulsifier and/or an emulsion stabiliser, in an amount from about 1 to about 30% by weight, preferably from about 1 to about 10%, for example, 1, 2, 3, 4, 5, 6, 7, 8, 9, or 10%, by weight, of the total composition.

Examples of suitable solubilising agents include sodium lauryl sulphate, polyethylene glycol 400, glycerol monostearate and castor oil and derivatives thereof such as polyethoxylated castor oil (for example, Cremphor® EL). In one embodiment of the invention, the composition comprises a solubilising agent or a mixture thereof in an amount from about 1 to about 40% by weight, such as 10 to 40% by weight, for example 10, 15, 20, 25, 30, 35 or 40% by weight, preferably about 20 to about 40% by weight, of the total composition.

Examples of suitable solvents include water, alcohols such as ethanol and/or polyols such as polyethylene glycol, propylene glycol and/or glycerol. In one embodiment of the invention, the solvent is an alcohol or a polyol, or a mixture thereof. In a preferred embodiment of the invention the solvent is selected from the group consisting of ethanol, glycerol, propylene glycol, polyethylene glycol (such as PEG 400) and mixtures thereof.

In another embodiment of the invention, the composition comprises a solvent in an amount from about 1 to about 60% by weight, preferably from about 20 to about 50% by weight, for example, 10, 15, 20, 25, 30, 35, 40, 45 or 50% by weight, of the total composition. In an alternative embodiment of the invention, the composition comprises less than about 60% by weight, typically less than 50%, suitably less than 40% (by weight of the total composition) of water. In a further embodiment of the invention the composition is substantially water-free.

Suitably, glycerol is present in the composition in an amount of from about 1 to about 5% (w/w), for example about 1, 2, 3, 4 or 5% (w/w), most preferably about 2% (w/w) (by weight of the total composition).

Suitably, ethanol is present in the composition in an amount of from about 1 to about 5% (w/w), for example about 1, 2, 3, 4 or 5% (w/w), most preferably about 2% (w/w) (by weight of the total composition).

Suitably, propylene glycol is present in the composition in an amount of from about 1 to about 20% (w/w), for example about 1, 2, 3, 4, 5, 10, 15 or 20% (w/w), most preferably about 2, 5 or 14% (w/w) (by weight of the total composition).

Suitably, polyethylene glycol is present in the composition in an amount of from about 10 to about 30% (w/w), for example about 10, 15, 20, 25 or 30% (w/w), most preferably about 20% (w/w) (by weight of the total composition).

Where water is present in the composition, it is typically present in an amount from about from about 10 to about 30% (w/w), for example about 10, 15, 20, 25 or 30% (w/w), most preferably about 10% (w/w) (by weight of the total composition). In one embodiment of the invention, there is provided a topical pharmaceutical composition comprising less than 60%, preferably less than 50%, of water and/or an aqueous buffer (such as citrate/phosphate pH 5.5 buffer) by weight of the total composition.

Examples of suitable thickening agents include hydroxyethylcellulose, hydroxypropylcellulose, carboxymethylcellulose sodium and/or metacrylic acid polymers and copolymers (such as carbomer) and polysaccharides (such as xanthan gum), and mixtures thereof. In one embodiment of the invention, the composition comprises a thickening agent in an amount from about 1 to about 50% by weight, preferably from about 10 to about 30% by weight, of the total composition.

Examples of suitable gelling agents include a polyoxyethylene-polyoxypropylene copolymer, glyceryl monooleate and glyceryl monostearate, and mixtures thereof. In one embodiment of the invention, the composition comprises a gelling agent in an amount from about 1 to about 30% by weight, preferably from about 1 to about 10% by weight, of the total composition.

Examples of suitable preservatives include benzyl alcohol, benzalkonium chloride, potassium sorbate and/or EDTA or salt thereof. In one embodiment of the invention, the composition comprises a preservative in an amount from about 1 to about 10% by weight, preferably from about 1 to about 5% by weight, of the total composition.

Suitably, benzyl alcohol is present is present in the composition in an amount of from about 0.1 to about 5% (w/w), for example about 0.25, 0.50, 1, 2, 3, 4 or 5% (w/w), most preferably about 0.5% (w/w) (by weight of the total composition).

In a preferred embodiment of the invention there is provided a topical pharmaceutical composition comprising 4-methyl-1-(2-phenylethyl)-8-phenoxy-2,3-dihydro-1H-pyrrolo[3,2-c]-quinoline or a pharmaceutically acceptable derivative thereof, benzyl alcohol, glycerol, ethanol, propylene glycol, polyethylene glycol (preferably PEG 400) and Unguentum M®.

In a more preferred embodiment of the invention there is provided a topical pharmaceutical composition comprising:
(i) 4-methyl-1-(2-phenylethyl)-8-phenoxy-2,3-dihydro-1H-pyrrolo[3,2-c]-quinoline or a pharmaceutically acceptable derivative thereof in an amount of from about 1 to about 2% (w/w) of the total weight of the composition;
(ii) benzyl alcohol in an amount of from about 0.1 to about 5% (w/w), preferably about 0.5% (w/w), of the total weight of the composition;
(iii) glycerol in an amount of from about 1 to about 5% (w/w), preferably about 2% (w/w), of the total weight of the composition;
(iv) ethanol in an amount of from about 1 to about 5% (w/w), preferably about 2% (w/w), of the total weight of the composition;
(v) propylene glycol in an amount of from about 1 to about 20% (w/w), preferably about 2 or about 15% (w/w), of the total weight of the composition;
(vi) PEG 400 in an amount from about 10 to about 30% (w/w), preferably about 20% (w/w) of the total weight of the composition; and
(vii) Unguentum M® in an amount from about 50 to about 75% (w/w), preferably from about 60 to about 70% (w/w) of the total weight of the composition.

In a further preferred embodiment of the invention there is provided a topical pharmaceutical composition comprising 4-methyl-1-(2-phenylethyl)-8-phenoxy-2,3-dihydro-1H-pyrrolo[3,2-c]-quinoline or a pharmaceutically acceptable derivative thereof, benzyl alcohol, propylene glycol, Emulsifying Ointment BP and liquid paraffin.

In a more preferred embodiment of the invention there is provided a topical pharmaceutical composition comprising:
(i) 4-methyl-1-(2-phenylethyl)-8-phenoxy-2,3-dihydro-1H-pyrrolo[3,2-c]-quinoline or a pharmaceutically acceptable derivative thereof in an amount of from about 1 to about 2% (w/w) of the total weight of the composition;
(ii) benzyl alcohol in an amount of from about 0.1 to about 5% (w/w), preferably about 2% (w/w), of the total weight of the composition;
(iii) propylene glycol in an amount of from about 1 to about 10% (w/w), preferably about 5% (w/w), of the total weight of the composition;
(iv) Emulsifying Ointment BP in an amount of from about 50 to about 75% (w/w), preferably about 65% (w/w), of the total weight of the composition; and
(v) liquid paraffin in an amount of from about 20 to about 40% (w/w), preferably about 30% (w/w) of the total weight of the composition.

In a further preferred embodiment of the invention, there is provided a topical pharmaceutical composition comprising 4-methyl-1-(2-phenylethyl)-8-phenoxy-2,3-dihydro-1H-pyrrolo[3,2-c]-quinoline or a pharmaceutically acceptable derivative thereof, water, petroleum jelly (preferably MEKUR® 773), a cholesterol derivative (preferably Softisan®) and castor oil or a derivative thereof (preferably Cremophor® EL).

In a more preferred embodiment of the invention there is provided a topical pharmaceutical composition comprising:
(i) 4-methyl-1-(2-phenylethyl)-8-phenoxy-2,3-dihydro-1H-pyrrolo[3,2-c]-quinoline or a pharmaceutically acceptable derivative thereof in an amount of from about 1 to about 15% (w/w) of the total weight of the composition;
(ii) water in an amount of from about 10 to about 30% (w/w), preferably about 10% (w/w) of the total weight of the composition;
(iii) petroleum jelly in an amount of from about 20 to about 75% (w/w), preferably about 35 to about 60% (w/w), of the total weight of the composition;
(iv) a cholesterol derivative in an amount of from about 20 to about 40% (w/w), preferably about 30% (w/w) of the total weight of the composition; and
(v) a solubilising agents, preferably castor oil or a derivative thereof, in an amount from about 1 to about 40% (w/w), preferably about 10 to about 40% (w/w) of the total weight of the composition.

In still a further preferred embodiment of the invention there is provided a topical pharmaceutical composition comprising 4-methyl-1-(2-phenylethyl)-8-phenoxy-2,3-dihydro-1H-pyrrolo[3,2-c]-quinoline or a pharmaceutically acceptable derivative thereof, preferably in an amount of about 15% (w/w), purified water, preferably in an amount of about 10% (w/w), MEKUR®773, preferably in an amount of about 35%, Softisan® 649, preferably in an amount of about 30% (w/w) and Cremophor® EL, preferably in an amount of about 10% (w/w). Preferably, said topical pharmaceutical composition is in the form of an ointment.

In still a further embodiment of the present invention there is provided a topical pharmaceutical composition comprising 4-methyl-1-(2-phenylethyl)-8-phenoxy-2,3-dihydro-1H-pyrrolo[3,2-c]-quinoline or a pharmaceutically acceptable derivative thereof, preferably in an amount of about 15% (w/w), purified water, preferably in an amount of about 10% (w/w), MEKUR®773, preferably in an amount of about 45%, and Softisan® 649, preferably in an amount of about 30% (w/w). Preferably, said topical pharmaceutical composition is in the form of an ointment.

The compositions of the invention are formulated for topical administration. The composition or medicament may be in the form of a cream, a lotion, an ointment, a spray, a gel, or a sterile aqueous solution or suspension. Preferably, the composition is in the form of a cream or ointment. More preferably, the composition is a cream or an ointment adapted for nasal administration, in particular for delivery to the anterior nares.

Such topical compositions may be prepared by any of the methods well known in the art of pharmacy e.g. as described in “Remington: The Science and Practice of Pharmacy”, Lippincott Williams and Wilkins, 21^{st} Edition, (2005), WO9510999, U.S. Pat. No. 6,974,585, WO2006048747, as well as in documents cited in any of these references.

Suitable methods include the step of admixing the active ingredient with a carrier which constitutes one or more excipients. For example, ointments and creams may be conveniently prepared by mixing together at an elevated temperature, such as 60-70° C., the components constituting the vehicle. The mixture may then be cooled to room temperature, and, after addition of any further ingredients, stirred to ensure adequate dispersion.

In one embodiment of the invention there is provided a process for preparing a topical pharmaceutical composition comprising 4-methyl-1-(2-phenylethyl)-8-phenoxy-2,3-dihydro-1H-pyrrolo[3,2-c]-quinoline or a pharmaceutically acceptable derivative thereof and a hydrophobic excipient, which process comprises the step of admixing 4-methyl-1-(2-phenylethyl)-8-phenoxy-2,3-dihydro-1H-pyrrolo[3,2-c]-quinoline or a pharmaceutically acceptable derivative thereof with a hydrophobic excipient.

The amount of active ingredients required for use in treatment will vary with the nature of the condition being treated and the age and condition of the patient, and will ultimately be at the discretion of the attendant physician or veterinarian. When administered in accordance with the present invention, 4-methyl-1-(2-phenylethyl)-8-phenoxy-2,3-dihydro-1H-pyrrolo[3,2-c]-quinoline or a pharmaceutically acceptable derivative thereof is typically present in an amount from about 0.1 to about 15%, for example, 0.1, 0.5, 1, 2, 3, 4, 5, 6, 7, 8, 9, 10, 11, 12, 13, 14 or 15% (w/w), such as from about 0.1 to about 5%, preferably 1 or 2%, by weight of the total composition.

The following examples illustrate topical pharmaceutical compositions of the present invention.

### EXAMPLES

In the following examples, “Compound (I)” means 4-methyl-1-(2-phenylethyl)-8-phenoxy-2,3-dihydro-1H-pyrrolo[3,2-c]-quinoline hydrochloride.

### Example 1

### Cream Formulation

| Component | % (w/w) |
|---|---|
| Compound (I) | 1 |
| Benzyl alcohol | 0.5 |
| Glycerol | 2 |
| Ethanol | 2 |
| Propylene glycol | 14 |
| PEG 400 | 20 |
| Unguentum M ® | 60.5 |

The propylene glycol, benzyl alcohol, glycerol, ethanol and PEG 400 were weighed into a suitably sized container and stirred until visually mixed. Subsequently, Compound (I) was weighed and added to the solvent mixture. The mixture was stirred until Compound (I) was visually observed to have dissolved. The Unguentum M® base was weighed into a separate suitably sized container and heated in a water bath set at 65° C. until it was visually observed to have melted. The solvent system containing Compound (I) was heated to between 60-65° C. and mixed with the heated Unguentum M® base and the formulation was homogenised. The formulation was stirred at ambient temperature using a PTFE magnetic follower until the viscosity increased, after which time the formulation was stirred by hand using a spatula.

### Example 2

### Cream Formulation

| Component | % (w/w) |
|---|---|
| Compound (I) | 1 |
| Benzyl alcohol | 0.5 |
| Glycerol | 2 |
| Ethanol | 2 |
| Propylene glycol | 2 |
| PEG 400 | 20 |
| Unguentum M ® | 72.5 |

Compound (I) was micronised using an Alpine 50 AS spiral jet mill to afford a D₅₀ particle size of <3 μm. The propylene glycol, benzyl alcohol, glycerol, ethanol and PEG 400 were weighed into a suitably sized container and stirred until visually mixed. Subsequently, the micronised Compound (I) was weighed and added to the solvent mixture. The mixture was stirred for 16 hours. The Unguentum M® base was weighed into a separate suitably sized container and heated in a water bath set at 65° C. until it was visually observed to have melted. The solvent system containing the dispersed Compound (I) was heated to 60-65° C. and mixed with the heated Unguentum M® and the formulation was homogenised. The formulation was stirred at ambient temperature using a PTFE magnetic follower until the viscosity increased, after which time the formulation was stirred by hand using a spatula.

### Example 3

### Cream Formulation

| Component | % (w/w) |
|---|---|
| Compound (I) | 2 |
| Benzyl alcohol | 0.5 |
| Glycerol | 2 |
| Ethanol | 2 |
| Propylene glycol | 14 |
| PEG 400 | 20 |
| Unguentum M ® | 59.50 |

Compound (I) was micronised using an Alpine 50 AS spiral jet mill to afford a D₅₀ particle size of <3 μm. The propylene glycol, benzyl alcohol, glycerol, ethanol and PEG 400 were weighed into a suitably sized container and stirred until visually mixed. Subsequently, the micronised Compound (I) was weighed and added to the solvent mixture. The mixture was stirred for 16 hours. The Unguentum M® base was weighed into a separate suitably sized container and heated in a water bath set at 65° C. until it was visually observed to have melted. The solvent system containing the dispersed Compound (I) was heated to 60-65° C. and mixed with the heated Unguentum M® and the formulation was homogenised. The formulation was stirred at ambient temperature using a PTFE magnetic follower until the viscosity increased, after which time the formulation was stirred by hand using a spatula.

### Example 4

### Ointment Formulation

| Component | % (w/w) |
|---|---|
| Compound (I) | 2 |
| Propylene glycol | 5 |
| Emulsifying Ointment BP | 63 |
| Liquid Paraffin | 30 |

Compound (I) was micronised using an Alpine 50 AS spiral jet mill to afford a D₅₀ particle size of <3 μm. The liquid paraffin, propylene glycol and micronised Compound (I) were weighed into a suitably sized container. The Compound (I) in liquid paraffin and propylene glycol were stirred at ambient temperature for 2 hours. The emulsified ointment BP was weighed into a separate suitably sized container and heated in a water bath set at 80° C. The emulsified ointment BP was heated until it was visually observed to have melted, after which time it was transferred to the Compound (I) in liquid paraffin and propylene glycol which had been heated to 60-65° C., and the container was stirred in a water bath set at 65° C. The formulation was stirred at ambient temperature using a PTFE magnetic follower until the viscosity increased, after which time the formulation was stirred by hand using a spatula.

### Example 5

### Ointment Formulation

| Component | % (w/w) |
|---|---|
| Compound (I) mesylate | 15 |
| Purified water | 10 |
| MERKUR ® 773 | 35 |
| Softisan ® 649 | 30 |
| Cremophor ® EL | 10 |

### Example 6

### Ointment Formulation

| Component | % (w/w) |
|---|---|
| Compound (I) mesylate | 15 |
| Purified water | 10 |
| MERKUR ® 773 | 45 |
| Softisan ® 649 | 30 |

Examples 5 and 6 may be prepared using analogous methods to those described in respect of Examples 1 to 4.

### Short term stability testing of formulations comprising 4-methyl-1-(2-phenylethyl)-8-phenoxy-2,3-dihydro-1H-pyrrolo[3,2-c]-quinoline mesylate and a hydrophobic excipient

Four compositions (A-D) were prepared as follows:

Composition A (300 g)—Placebo

| | Composition | | |
|---|---|---|---|
| Active/Excipient | Target % | Target (g) | Range (g) |
| Compound (I) | 0 | — | — |
| mesylate | | | |
| Purified water | 10 | 30 | 29.50-30.50 |
| MERKUR ® 773 | 50 | 150 | 149.50-151.50 |
| Softisan ® 649 | 30 | 90 | 89.50-91.50 |
| Cremophor ® EL | 10 | 30 | 29.50-30.50 |

Composition B (250 g)—15% Active Ingredient

| | Composition | | |
|---|---|---|---|
| Active/Excipient | Target % | Target (g) | Range (g) |
| Compound (I) | 15 | 37.5 | 37.40-37.60 |
| mesylate | | | |
| Purified water | 10 | 25 | 49.50-50.50 |
| MERKUR ® 773 | 35 | 87.5 | 87.00-88.00 |
| Softisan ® 649 | 30 | 75 | 74.50-75.50 |
| Cremophor ® EL | 10 | 25 | 24.50-25.50 |

Composition C (300 g)—Placebo

| | Composition | | |
|---|---|---|---|
| Active/Excipient | Target % | Target (g) | Range (g) |
| Compound (I) | 0 | — | — |
| mesylate | | | |
| Purified water | 10 | 30 | 29.50-30.50 |
| MERKUR ® 773 | 60 | 180 | 179.50-181.50 |
| Softisan ® 649 | 30 | 90 | 89.50-91.50 |
| Cremophor ® EL | 0 | — | — |

Composition D (250 g)—15% Active Ingredient

| | Composition | | |
|---|---|---|---|
| Active/Excipient | Target % | Target (g) | Range (g) |
| Compound (I) | 15 | 37.5 | 37.40-37.60 |
| mesylate | | | |
| Purified water | 10 | 25 | 24.50-25.50 |
| MERKUR ® 773 | 45 | 112.5 | 112.0-113.0 |
| Softisan ® 649 | 30 | 75 | 74.50-75.50 |
| Cremophor ® EL | 0 | — | — |

Preparative Methods

(1) Compositions A and B

The target quantities of Softisan® 649 and MERKUR® 773 were weighed directly into a 600 mL beaker, heated on a hotplate/stirrer at 75-85° C. and mixed using a magnetic stirrer bar for approximately 90 minutes until a clear melt was observed (then held at 75-80° C.). The required quantity of Compound (I) mesylate (where present) was weighed directly into a 150 mL borosilicate glass beaker, followed by 80% of the purified water quantity. The beaker was covered with aluminium foil to minimise evaporation and mixed using a magnetic stirrer bar for approximately 5 minutes (a suspension was formed). The remaining 20% of the purified water was weighed into a 7 mL glass vial, sealed with a screw cap and heated to 75-85° C. The suspension containing Compound (I) mesylate was heated to 75-80° C. on a hotplate/stirrer until the solution was observed to become clear (complete dissolution of Compound (I) mesylate) and then removed from the heat source. The required quantity of Cremophor® EL was added into the beaker containing Compound (I) and homogenised immediately using a pre-warmed small mixing head and Silverson L4RT homogeniser at maximum speed (10,600 rpm). Homogenisation continued for 2 minutes until a visually homogeneous solution was evident. The beaker containing the Compound (0/water/Cremophor®EL solution was returned to the hotplate and re-heated to 75-80° C. This solution was added to the melted Softisan® 649 and MERKUR® 773 under stirring at 75-80° C. and the beaker was washed out using the remaining pre-heated purified water (20% of the target quantity). The beaker containing all of the excipients and compound (I) was homogenised at 10,600 rpm for 2 minutes using a pre-warmed intermediate mixing head. The formulation was stirred continuously using a Heildoph mixer (set at 250 rpm) and stainless steel paddle until it reached ambient temperature (15-25° C.), with intermittent mixing using a palette knife.

(2) Compositions C and D

The target quantities of Softisan® 649 and MERKUR® 773 were weighed directly into a 600 mL beaker, heated on a hotplate/stirrer at 75-85° C. and mixed using a magnetic stirrer bar for approximately 90 minutes until a clear melt was observed (then held at 75-80° C.). The required quantity of Compound (I) mesylate was weighed directly into a 150 mL borosilicate glass beaker, followed by 80% of the purified water quantity. The beaker was covered with aluminium foil to minimise evaporation and mixed using a magnetic stirrer bar for approximately 5 minutes (a suspension was formed). The remaining 20% of the purified water was weighed into a 7 mL glass vial, sealed with a screw cap and heated to 75-85° C.

The suspension containing Compound (I) mesylate was heated to 75-80° C. on a hotplate/stirrer until the solution was observed to become clear (complete dissolution of Compound (I) mesylate) and then removed from the heat source. This solution was added to the melted Softisan® 649 and MERKUR® 773 under stirring at 75-80° C. and the beaker was washed out using the remaining pre-heated purified water (20% of the target quantity). The beaker containing all of the excipients and compound (I) was homogenised at 10,600 rpm for 2 minutes using a Silverson L4RT homogeniser equipped with a pre-warmed intermediate mixing head. The formulation was then stirred continuously at 120 rpm for approximately 2 hours 30 minutes using a Heidolph mixer and stainless steel paddle until it reached ambient temperature (15-25° C.), with intermittent mixing using a palette knife.

Each of the four compositions were hand-filled into white aluminium screw-cap tubes (Lindhardt GmbH) and amber borosilicate glass (screw-cap) vials. The filing procedure was performed as follows: the composition was dispensed into a polypropylene syringe using a spatula and a minimum of 1.35 g (target range 1.35-1.45 g) was transferred into each tube or glass vial. The tubes were hand-crimped to seal and the vial caps were applied and sealed with Parafilm®. The samples were stored at ambient temperature (15-25° C.) prior to stability testing.

Stability Testing

In addition to the allowance for analysis at T=0, sufficient tubes and glass vials containing the four compositions were placed onto stability at 2-8° C., 25° C./60% relative humidity (RH), 30° C./65% RH and 40° C./75% RH to allow for sufficient testing at T=2 and 4 weeks. The testing schedule is detailed in the table below.

| | | Time point in weeks | |
|---|---|---|---|
| Storage condition | Initial (T = 0) | 2 | 4 |
| 2-8° C. | X | X* | X* |
| 25° C./60% RH | X | X | X |
| 30° C./65% RH | X | X* | X* |
| 40° C./75% RH | X | X | X |

X: testing performed
X*: testing performed only if degradation was observed at the immediately higher storage condition.

Compound (I) Identification, Content and Impurities

Compound (I) mesylate identification was performed by overlaying a 150 μg/mL Compound (I) mesylate standard solution and the active product chromatogram. To conform to the specification the difference in retention time between the two Compound (I) mesylate peaks should be no greater than ±10% of the standard solution retention time.

The chromatographic conditions for the analysis of Compound (I) mesylate are given in the table below:

| HPLC system | Waters 2695 Separations Module and | | |
|---|---|---|---|
| | Waters ™ 996/2996 photodiode array | | |
| | detector in conjunction with Empower Pro 2 | | |
| Column | Zorbax Eclipse XDB-C18, 5 μm, 4.6 × 150 mm | | |
| | (Serial number: USKH070493) | | |
| Detection | 254 nm | | |
| Guard column: | Phenomenex, C18, 4 × 2.0 mm, Cat. No.: AJ0-4286 | | |
| Sample Temperature | 5 ± 3° C. | | |
| Column Temperature | 40 ± 2° C. | | |
| Flow Rate | 1 mL/min | | |
| Mobile Phase | Mobile phase A: 0.01M ammonium acetate | | |
| | adjusted to pH 8 using ammonium hydroxide | | |
| | (Section 6.1.1) | | |
| | Mobile phase B: Acetonitrile (Section 6.1.2) | | |
| Gradient | Time (min) | Line A (%) | Line B (%) |
| | 0 | 95 | 5 |
| | 5 | 95 | 5 |
| | 15 | 5 | 95 |
| | 25 | 5 | 95 |
| | 27 | 95 | 5 |
| | 32 | 95 | 5 |

| | |
|---|---|
| Injection Volume | 10 μL |
| Run Time | 32 min |
| Needle wash | 80:20 Acetonitrile:water |
| Seal wash | 60:40 Acetonirile:water |

Compound (I) mesylate content was determined as follows: at T=0, n=3 extractions were performed from the top, middle and bottom of the mixing vessel for the bulk active products and n=1 extraction was performed from each of three filled active tubes (selected from the start, middle and end of the filling run). For the placebo compositions, a single extraction was performed from the middle of the mixing vessel for the bulk product and a single extraction from the middle of the fill tube. For the active ingredients, at each subsequent time point two extractions were performed from the upright and inverted tubes, while n=3 extractions were performed from the glass vials. A single extraction was performed for each placebo composition. In addition, at T=2 weeks (25° C./60% RH only) and T=4 weeks (2-8° C., 25° C./60% RH, 40° C./75% RH) the complete contents of a single tube of active ingredient (approximately 1 g) was dispensed onto a 90 mm plastic Petri-dish and manually mixed for 30 seconds using a spatula prior to performing n=3 random extractions.

Compound (I) mesylate impurities were determined by overlaying the respective active and placebo product chromatograms and that of a blank solution (acetonitrile). Any peaks present in the active product that were not evident in the placebo or blank were integrated on a % a/a basis of the main compound (I) mesylate peak.

Results

The visual appearance (both macroscopic and microscopic) of compositions A-D remained unchanged at all tested storage conditions up to the 4 week stability time point, with the exception of composition D at T=4 weeks where a slight darkening was observed at 40° C./75% RH due to oxidation.

Compound (I) mesylate identification, content and impurities were determined in accordance with the methods given above. All four compositions were observed to meet the specification for Compound (I) mesylate identification at T=0, 2 and 4 weeks at all tested stability storage conditions.

Conclusions

Batches of the four compositions A-D were placed onto stability at the ICH recommended storage conditions of 2-8° C., 25° C./60% RH, 30° C./65% RH and 40° C./75% RH for 4 weeks.

Based upon the data obtained during the 4 week stability testing of the compositions, it can be concluded that all of the products were chemically stable at ° C.

Release, Activity and Residue Activity Tests of Examples 5 and 6 Against Staphylococcus aureus on Pig Skin

Bacterial strain used: Staphylococcus aureus (Oxford); Gram positive; Reference strain.

Bacterial Growth Conditions

S. aureus was grown in 10 ml of nutrient broth No. 2 (Oxoid) overnight at 37° C. with continuous shaking at 120 rpm.

Antibiotic Compositions

Ointment compositions (B) and (D) comprising 15% (w/w) of Compound (I) mesylate were prepared in accordance with Examples 5 and 6. Corresponding placebo compositions (A) and (C) were also prepared.

| | Theoretical composition (%, w/w) | | | |
|---|---|---|---|---|
| | Composition | Composition | Composition | Compo- |
| | A | B | C | sition D |
| Active/Excipient | (Placebo) | (Example 5) | (Placebo) | (Example 6) |
| Compound (I) | 0 | 15 | 0 | 15 |
| mesylate | | | | |
| Purified water | 10 | 10 | 10 | 10 |
| MERKUR ® 773 | 50 | 35 | 60 | 45 |
| Softisan ® 649 | 30 | 30 | 30 | 30 |
| Cremophor ® EL | 10 | 10 | 0 | 0 |

Test Conditions

The activities of the compositions (A-D) were tested against S. aureus on pig skin. The skin was washed twice in sterile distilled water. After washing, the skin was placed into a petri dish and cut into about 2 cm² pieces. The fat at the back of the skin was removed with scissors. The bacterial cultures (20 to 25 μl) were spread onto the skin. The bacteria were allowed to dry for about 10 minutes. The formulations (45 to 70 μl) were added on to the skin to cover the bacterial cells. The skin was incubated at 33-35° C. for different time points up to 24 hours.

Results

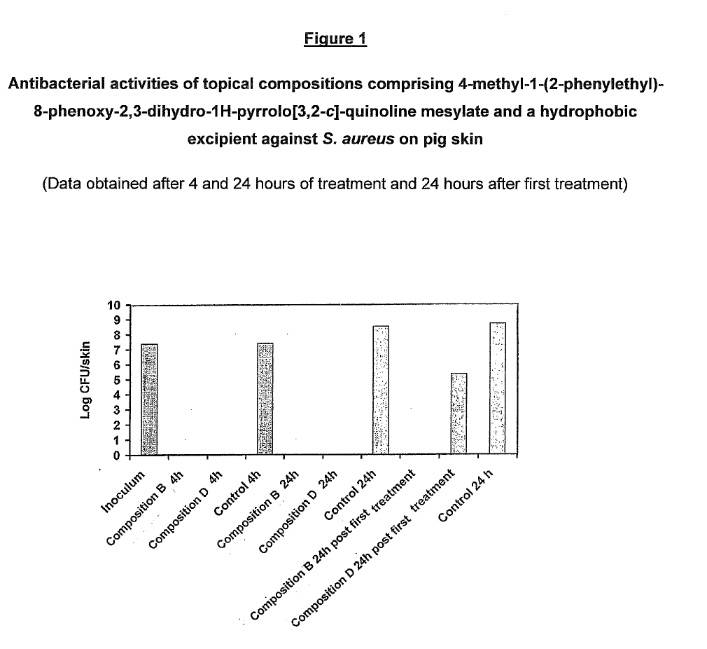

The results obtained are summarised in FIG. 1.

Conclusions

Compositions B and D showed complete kill of bacteria after 4 and 24 hours of treatment. 24 hours after the first 4 hour treatment, CFU counts recovered from the Composition D treated skin samples, but no CFU counts recovered from the skin treated with Composition B. No significant reduction in antibacterial effect was observed for either of the test compositions after storage at ambient conditions for 2.5 months.

1. A topical pharmaceutical composition comprising 4-methyl-1-(2-phenylethyl)-8-phenoxy-2,3-dihydro-1H-pyrrolo[3,2-c]-quinoline or a pharmaceutically acceptable derivative thereof and a hydrophobic excipient.

2. A composition according to claim 1, which exhibits a surface residence time of greater than 15 minutes, preferably greater than 30 minutes, following application to the skin or mucosal surface.

3. A composition according to claim 1, wherein the hydrophobic excipient is a paraffin-based excipient, or an ointment or cream base containing a paraffin-based excipient.

4. A composition according to claim 3, wherein the paraffin-based excipient is selected from the group consisting of mixtures of solid and/or semi-solid saturated hydrocarbons having the general formula CₙH₂ₙ₊₂ obtainable from petroleum and/or shale oil, paraffin, white soft paraffin, liquid paraffin, light liquid paraffin and petrolatum, and mixtures thereof.

5. A composition according to claim 3, wherein the ointment or cream base containing a paraffin-based excipient is selected from the group consisting of Unguentum M®, Paraffin Ointment BP, Simple Ointment BP and Emulsifying Ointment BP, and mixtures thereof.

6. A composition according to claim 1, wherein the hydrophobic excipient is selected from the group consisting a “fixed” (vegetable based) oil or a hydrogenated derivative thereof, a cholesterol derivative, and a fatty acid, and mixtures thereof.

7. A composition according to claim 1, which comprises from about 25 to about 99% (by weight of the total composition) of one or more hydrophobic excipients.

8. A composition according to claim 7, which comprises from about 50 to about 75%, (by weight of the total composition) of one or more hydrophobic excipients.

9. A composition according to claim 1, comprising one or more additional excipients selected from the group consisting of emulsifiers, solubilising agents, solvents, thickening agents, gelling agents, and/or preservatives.

10. A composition according to claim 1, wherein 4-methyl-1-(2-phenylethyl)-8-phenoxy-2,3-dihydro-1H-pyrrolo[3,2-c]-quinoline or a pharmaceutically acceptable derivative thereof is present in an amount from about 0.1 to about 15% by weight of the total composition.

11. A composition according to claim 10, wherein 4-methyl-1-(2-phenylethyl)-8-phenoxy-2,3-dihydro-1H-pyrrolo[3,2-c]-quinoline or a pharmaceutically acceptable derivative thereof is present in an amount of 1 or 2% by weight of the total composition.

12. A composition according to claim 1, wherein the composition is in the form of a cream or an ointment.

13. A composition according to claim 12, wherein the cream or ointment is adapted for nasal administration.

14. A composition according to claim 1, comprising 4-methyl-1-(2-phenylethyl)-8-phenoxy-2,3-dihydro-1H-pyrrolo[3,2-c]-quinoline hydrochloride or 4-methyl-1-(2-phenylethyl)-8-phenoxy-2,3-dihydro-1H-pyrrolo[3,2-c]-quinoline methane sulfonate.

15. A process for preparing a composition according to claim 1, which process comprises the step of admixing 4-methyl-1-(2-phenylethyl)-8-phenoxy-2,3-dihydro-1H-pyrrolo[3,2-c]-quinoline or a pharmaceutically acceptable derivative thereof with a hydrophobic excipient.

16. Use of a topical pharmaceutical composition comprising 4-methyl-1-(2-phenylethyl)-8-phenoxy-2,3-dihydro-1H-pyrrolo[3,2-c]-quinoline or a pharmaceutically acceptable derivative thereof and a hydrophobic excipient for the treatment of a microbial infection.
